(19) United States
(12) Patent Application Publication
(10) Pub. No.: US 2018/0200308 A1
(43) Pub. Date: Jul. 19, 2018

(54) METHODS OF TREATING COLITIS

(71) Applicant: Seres Therapeutics, Inc., Cambridge, MA (US)

(72) Inventors: David COOK, Brookline, MA (US); Matthew HENN, Belmont, MA (US); Jennifer LACHEY, Arlington, MA (US)

(21) Appl. No.: 15/742,732

(22) PCT Filed: Jul. 8, 2016
(86) PCT No.: PCT/US16/41538

§ 371 Date: Jan. 8, 2018

Related U.S. Application Data
(60) Provisional application No. 62/190,123, filed on Jul. 8, 2015.
(60) Provisional application No. 62/250,829, filed on Nov. 4, 2015.

(51) Int. Cl. A61K 35/742; A61K 38/14; A61K 9/20; A61K 9/48; A61P 1/00

(52) U.S. Cl. CPC A61K 35/742; A61K 38/14; A61K 2300/00; A61K 9/48; A61P 1/00; A61K 9/20

(57) ABSTRACT

Provided are methods of treating a colitis using a microbial composition and antibiotic.

### FIELD OF THE INVENTION

The application relates to methods of treating inflammatory bowel disease using microbiome related technologies.

### BACKGROUND OF THE INVENTION

Colitis is a condition involving inflammation of the colon and includes inflammatory bowel disease (IBD). IBD is characterized by relapsing and remitting signs and symptoms and chronic inflammation at various sites in the gastrointestinal (GI) tract. Crohn's disease and ulcerative colitis (UC) are examples of IBD. Symptoms of IBD typically result in diarrhea and abdominal pain. According the Merck Manual “[n]o specific environmental, dietary, or infectious causes have been identified” (Walfish and Sachar, 2012, merckmanuals.com/professional).

Crohn's disease typically, although not always, involves the small bowel. Patients commonly develop fistulas, masses, and abscesses, and may have significant perianal lesions. Ulcerative colitis is typically confined to the colon and involves the rectosigmoid. Gross rectal bleeding always occurs in ulcerative colitis patients, however these patients do not develop fistulas or significant perianal lesions. Treatment for IBD can include, for example, supportive care, 5-aminosalicylic acid and derivatives, corticosteroids, immunomodulators, cytokines, antibiotics, and probiotics, for example, non-pathogenic E. coli, Lactobacillus species and Saccharomyces, which may be effective in preventing pouchitis, but other therapeutic roles have not been clearly defined (Merck Manual, supra). Human fecal transplant has reportedly produced positive results in some cases.

In view of the inadequate treatments available, improved methods of treating colitis are needed, including improved methods that have increased efficacy compared to standard of care treatments and/or have similar efficacy to standard of care with reduced risk of undesirable side effects and/or increase the efficacy of the standard of care.

### SUMMARY OF THE INVENTION

The invention relates to the discovery that colitis can be treated using a combination of a bacterial spore composition and an antibiotic.

Accordingly, the invention provides methods of treating subjects (e.g., human subjects) diagnosed with a colitis (e.g., IBD, such as, for example, Crohn's disease or ulcerative colitis). The methods include treating, for example, subjects who have active colitis and/or subjects who have been diagnosed with mild to moderate ulcerative colitis. The methods include (a) administering an antibiotic (e.g., vancomycin) to the subject, and (b) administering a bacterial spore composition (BSC) to the subject.

In certain embodiments, the antibiotic and the bacterial spore composition are administered concurrently, while in other embodiments the antibiotic and the bacterial spore composition are administered sequentially.

In various examples, the bacterial spore composition is optionally administered within 24 hours, 2 days, 3 days, 4 days, 5 days, 6 days, 7 days, 10 days, two weeks, or three weeks of the dosing (e.g., the final dosing) of the antibiotic.

The bacterial spore composition can optionally be administered in a single dose, or in multiple doses.

Furthermore, the bacterial spore composition can optionally be administered every day, at least every other day, at least every 3 days, at least every 4 days, at least every 5 days, at least every 6 days, at least every week, at least every two weeks, at least every 3 weeks, at least every 4 weeks, at least every 8 weeks, at least every 12 weeks, or at least every 16 weeks.

In certain embodiments, the subject is treated with the BSC weekly for at least 8 weeks or daily for at least 8 weeks.

In various embodiments, the bacterial spore composition is in a capsule or a pill.

In other embodiments, the composition includes less than or equal to 99% vegetative cells (e.g., less than or equal to 20% vegetative cells).

The bacterial spore composition can include spore forming bacteria and/or spores. In various examples, the spores are directly derived from human feces, e.g., by the use of ethanol. In some embodiments, the composition consists essentially of spores.

The invention also provides the use of a bacterial spore composition, in combination with an antibiotic, for treating colitis (e.g., as described herein), as well as the use of a bacterial spore composition, in combination with an antibiotic, for preparing medicaments for treating colitis (e.g., as described herein).

The entire disclosure of each patent document and scientific article referred to herein, and those patent documents and scientific articles cited thereby, is expressly incorporated by reference herein for all purposes.

Additional features and advantages of the invention are more particularly described below.

### BRIEF DESCRIPTION OF THE DRAWINGS

FIG. 1 is a graph showing the effect of a bacterial spore composition (BSC) on DSS-induced changes in colon length.

FIG. 2 is a graph showing the effect of a BSC on colon gross pathology score in the DSS model of colitis.

FIG. 3 is a graph showing the effect of a BSC on maximum DSS-induced body weight change.

FIG. 4 is a graph showing the effect of a BSC on clinical score in the DSS model of colitis.

### DETAILED DESCRIPTION OF THE INVENTION

The therapeutic compositions and methods provided herein are useful for the treatment of colitis. Also provided are methods useful for developing colitis treatments that have improved efficacy and a low incidence of adverse events compared to the standard of care. In addition, non-immunosuppressive therapeutic compositions and methods for treating inflammatory bowel disease (IBD) are provided. As used herein, a “therapeutic composition” comprises a microbial composition, e.g., a bacterial spore composition (BSC) and optionally an antibiotic, which may be administered together or separately in form and/or time. In some embodiments, the treatment is with a BSC without antibiotic.

In some embodiments, the methods described herein include administration of an antibiotic to a patient diagnosed with a colitis, and a microbial composition, e.g., a microbial composition derived from feces (e.g., containing vegetative bacteria and spores or substantially containing only spores) or a designed composition comprising selected bacteria. In some embodiments, at least some of the selected bacteria are capable of forming spores. In some embodiments, the bacteria are substantially in spore form and the microbial composition is termed herein a bacterial spore composition (BSC).

The invention also includes the use of a BSC, as described herein, in combination with an antibiotic, as described herein, for treating colitis (e.g., IBD, such as Crohn's disease or ulcerative colitis), and the use of these agents for preparing medicaments for such treatment.

Microbial Compositions

The microbial compositions used in the invention include bacterial spore compositions (BSCs). In addition to spore forming bacteria (whether in the form of spores and/or in vegetative form), such compositions can also optionally include non-spore forming bacteria (e.g., Lactobacillus, Bacteroides, or Bifidobacterium). Thus, in some embodiments microbial compositions suitable for use in the present invention are bacterial compositions substantially composed of spores (spore compositions) or spore forming bacteria, for example, containing greater than or equal to 1% spores, greater than or equal to 5% spores, greater than or equal to 10% spores, greater than or equal to 20% spores, greater than or equal to 50% spores, greater than or equal to 80% spores, greater than or equal to 85% spores, greater than or equal to 90% spores, greater than or equal to 95% spores, greater than or equal to 98% spores, greater than or equal to 99% spores, or equal to 100% spores. Spore content can be determined using methods known in the art, for example, using a dipicolinic (DPA) assay, spore CFU assay, or a combination of such assays. In some embodiments, the percentage of spores refers to the percentage of germinable bacterial spores in a composition. Percentage of spores can further refer to percent biomass (w/w), number of total organisms, e.g., number of viable organisms detected using methods known in the art. Percentage spores can also be referred to by the number of genomes detected.

Of note, spores provide a convenient formulation because spores are resistant to oxygen and gastric acid. Similar effects can be obtained by spore compositions in which the predominant biomass (e.g., 51%, 60%, 70%, 80%, 90%, 95%, 99%, or greater) comprises vegetative forms of these spore forming organisms.

Microbial compositions, e.g., spore compositions useful in the invention include spore preparations derived from fecal material, purified preparations of microbiota from fecal material, or spore formulations, for example, prepared from cultured bacteria in a spore form. In various embodiments, the spore preparations are made from human fecal material, such as human fecal material obtained from healthy human donors. In certain examples, fractions including bacterial cells and spores from such human fecal material is treated with a solvent (e.g., ethanol, such as 50% ethanol, wt/wt), to generate a composition including Firmicutes spores. Examples of such compositions are provided in, for example, PCT/US2014/014745 (WO 2014/121302) and Example 1, infra.

Spore Assays

Methods of determining the spore content of a composition are known in the art. For example, a dipicolinic assay (DPA assay), which uses fluorescence monitoring of DPA release upon heat inactivation of spores based on enhanced fluorescence of the terbium ion upon binding to DPA (e.g., see Rosen et al., 1997, Anal Chem 69:1082-1085) can be used.

Microbiological assay methods that are known in the art are useful for determining the spore content of a composition. In general, such assays involve treating a composition under conditions that kill vegetative cells (e.g., heat or an appropriate solvent), plating the resulting spores under conditions favorable for germination and growth, and determining the number of spores and/or diversity of spores based on the colony forming units (CFUs). Generally, the number of spore CFUs is always less than the total number of spores because germination is an inherently stochastic process and the entire population does not germinate synchronously. Similar procedures and growth media, without the use of solvent or heat inactivation, can be used to quantify the total CFU content including spore forming vegetative organisms that may be present in a composition.

Antibiotics

In certain embodiments of methods described herein, an antibiotic therapy is administered prior to, concurrently with, both prior to and concurrently with, after, or both concurrently with or after a microbial composition (BSC). Examples of useful antibiotics include, for example, vancomycin, neomycin, rifaximin, metronidazole, and fidaxomicin. In general, the antibiotic is not one associated with causation of Clostridium infection, e.g., fluoroquinolones, cephalosporins, clindamycin, and penicillins.

Dose, Formulation, Delivery

Methods useful in the invention include, for example, administration of combinations of a BSC and one or more antibiotics (termed a “therapeutic combination”). In general, treatment of a subject that has colitis with a therapeutic combination results in improvement in at least one sign or symptom of the disease, an improvement in the duration of the improvement of at least one sign or symptom of the disease, or a decrease in at least one side effect or adverse event such as those generally attributable to treatment with an antibiotic alone (or BSC alone).

Dose

The number of spores in a BSC dose is generally 10⁴ to 10⁹. In some embodiments, the dose is, for example, 10⁵ to 10⁹, 10⁶ to 10⁹, 10⁶ to 10⁸, 10⁷ to 10⁹, 10⁸ to 10⁹, 10⁶, 10⁷, 10⁸, 10⁹, or 10¹⁰, or any range between one of these values.

The antibiotic component of a therapeutic combination is generally provided in a standard dose, as is known in the art.

The dosing regimen generally includes providing the therapeutic combination daily, every two days, every three days, every four days, every five days, weekly, every two weeks, every three weeks or monthly. Treatment can be chronic or for a limited time, e.g., in response to a colitis attack.

Formulation

Spores are formulated based on their method of delivery and storage. For example, spores for delivery via enema, rectal tube, nasogastric tube, gastroscope, or colonoscope are typically in a pharmaceutically acceptable liquid. Examples of methods for preparing a BSC can be found in PCT/US2014/014745 (WO 2014/121302).

Delivery

Therapeutic compositions or separate components of such compositions (e.g., BSC or antibiotic) can be delivered using methods known in the art, for example, orally (e.g., in a capsule), via colonoscope to the proximal colon, by enema/rectal tube to the distal lower GI tract, by nasogastric tube/gastroscope to the upper GI tract, in a capsule, or pill. Delivery may be targeted to a known or suspected disease site. Antibiotics can be delivered by additional suitable methods, e.g., injection or infusion.

Additional details regarding dose, formulation, and delivery of, e.g., BSC compositions, are as follows.

Compositions described herein can be prepared and administered using methods known in the art, including local administration and systemic administration routes suitable for the type of composition as described supra. For example, microbial compositions are typically administered orally or directly to the gastrointestinal tract whereas an antibiotic therapeutic that is a component of a method may be delivered orally, directly to the gastrointestinal tract, by infusion, injection, inhalation, or other method used in the art.

Components of a therapeutic composition may be formulated using conventional pharmaceutical carriers, aqueous, powder or oily bases, thickeners and the like.

In some embodiments, the pharmaceutical compositions comprise, as the active ingredient, one or more of the agents above in combination with one or more pharmaceutically acceptable carriers (excipients). In making a therapeutic compositions or component thereof, the agent is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the formulations can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

A composition can be formulated in a unit dosage form. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Components of a therapeutic composition can be provided in a kit with instructions for administering the components.

A capsule, tablet or pill comprising a composition can be coated or otherwise compounded to provide a dosage form affording the advantage of prolonged action or release in the desired section of the gastrointestinal tract, e.g., in the colon. For example, a BSC can be provided in a capsule can comprise an inner and an outer component, the latter being in the form of an envelope over the former. Suitable materials for such capsules include, for example, hypermellose.

A liquid formulation comprising a bacterial composition can be prepared for oral delivery, for example, in an aqueous solutions, suitably flavored syrups, aqueous or oil suspensions, or flavored emulsions with edible oils such as cottonseed oil, sesame oil, coconut oil, or peanut oil, as well as elixirs and similar pharmaceutical vehicles.

The amount and frequency of a composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications, i.e., ameliorate disease. Effective doses will depend on the disease condition being treated as well as by the judgment of the attending clinician depending upon factors such as the severity of the disease, the age, weight and general condition of the patient, and the like. The therapeutic dosage of a composition can vary according to, for example, the particular use for which the treatment is made, the manner of administration of the composition, the health and condition of the patient, and the judgment of the prescribing physician.

Additional information regarding dose, formulation, and delivery of compositions according to the invention is as follows. In some examples, a composition is administered as a pharmaceutical preparation in solid, semi-solid, micro-emulsion, gel, or liquid form. Examples of such dosage forms include tablet forms disclosed in U.S. Pat. Nos. 3,048,526, 3,108,046, 4,786,505, 4,919,939, and 4,950,484; gel forms disclosed in U.S. Pat. Nos. 4,904,479, 6,482,435, 6,572,871, and 5,013,726; capsule forms disclosed in U.S. Pat. Nos. 4,800,083, 4,532,126, 4,935,243, and 6,258,380; and liquid forms disclosed in U.S. Pat. Nos. 4,625,494, 4,478,822, and 5,610,184; each of which is incorporated herein by reference in its entirety. Forms of the compositions that can be used orally include tablets, push-fit capsules made of gelatin, as well as soft, sealed capsules made of gelatin and a plasticizer, such as glycerol or sorbitol. Tablets can be made by compression or molding, optionally with one or more accessory ingredients.

Compressed tablets can be prepared by compressing the active ingredient in a suitable machine, in a free-flowing form such as a powder or granules, optionally mixed with binders (e.g., povidone, gelatin, hydroxypropylmethyl cellulose), inert diluents, preservative, antioxidant, disintegrant (e.g., sodium starch glycolate, cross-linked povidone, cross-linked sodium carboxymethyl cellulose) or lubricating, surface active or dispersing agents. Molded tablets can be made by molding in a suitable machine a mixture of the powdered compound moistened with an inert liquid diluent. The tablets can optionally be coated or scored and can be formulated so as to provide slow or controlled release of the active ingredient therein. Tablets can optionally be provided with an enteric coating, to provide release in stomach or in parts of the gut (e.g., colon, lower intestine) other than the stomach. All formulations for oral administration can be in dosages suitable for such administration. The push-fit capsules can contain the active ingredients in admixture with filler, such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. Dragee cores are provided with suitable coatings. For this purpose, concentrated sugar solutions can be used, which can optionally contain gum arabic, talc, polyvinyl pyrrolidone, carbopol gel, polyethylene glycol, or titanium dioxide, lacquer solutions, and suitable organic solvents or solvent mixtures. Dyestuffs or pigments can be added to the tablets or Dragee coatings for identification or to characterize different combinations of active compound doses. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water soluble carrier such as polyethylene glycol or an oil medium, for example peanut oil, liquid paraffin, or olive oil. Oral liquid preparations can be in the form of, for example, aqueous or oily suspensions, solutions, emulsions syrups or elixirs, or can be presented as a dry product for reconstitution with water or other suitable vehicle before use. Such liquid preparations can contain conventional additives, such as suspending agents, for example sorbitol, methyl cellulose, glucose syrup, gelatin, hydroxyethyl cellulose, carboxymethyl cellulose, aluminum stearate gel or hydrogenated edible fats, emulsifying agents, for example lecithin, sorbitan monooleate, acacia; nonaqueous vehicles (which can include edible oils), for example almond oil, oily esters such as glycerine, propylene glycol, or ethyl alcohol; preservatives, for example methyl or propyl p-hydroxybenzoate or sorbic acid, and, if desired, conventional flavoring or coloring agents.

In one embodiment, a provided bacterial spore composition includes a softgel formulation. A softgel can contain a gelatin based shell that surrounds a liquid fill. The shell can be made of gelatin, plasticizer (e.g., glycerin and/or sorbitol), modifier, water, color, antioxidant, or flavor. The shell can be made with starch or carrageenan. The outer layer can be enteric coated. In one embodiment, a softgel formulation can include a water or oil soluble fill solution, or suspension of a composition, for example, a bacterial spore composition, covered by a layer of gelatin.

An enteric coating can control the location of where a bacterial spore composition is absorbed in the digestive system. For example, an enteric coating can be designed such that a bacterial spore composition does not dissolve in the stomach but rather travels to the small intestine, where it dissolves. An enteric coating can be stable at low pH (such as in the stomach) and can dissolve at higher pH (for example, in the small intestine). Material that can be used in enteric coatings includes, for example, alginic acid, cellulose acetate phthalate, plastics, waxes, shellac, and fatty acids (e.g., stearic acid, palmitic acid). Enteric coatings are described, for example, in U.S. Pat. Nos. 5,225,202, 5,733,575, 6,139,875, 6,420,473, 6,455,052, and 6,569,457, all of which are herein incorporated by reference in their entirety. The enteric coating can be an aqueous enteric coating. Examples of polymers that can be used in enteric coatings include, for example, shellac, cellulose acetate phthalate, polyvinylacetate phthalate, and methacrylic acid. Enteric coatings can be used to (1) prevent the gastric juice from reacting with or destroying the active substance, (2) prevent dilution of the active substance before it reaches the intestine, (3) ensure that the active substance is not released until after the preparation has passed the stomach, and (4) prevent live bacteria contained in the preparation from being killed because of the low pH-value in the stomach. In one embodiment a bacterial spore composition or the bacterial component of a food or beverage is provided as a tablet, capsule, or caplet with an enteric coating. In one embodiment the enteric coating is designed to hold the tablet, capsule, or caplet together when in the stomach. The enteric coating is designed to hold together in acid conditions of the stomach and break down in non-acid conditions and therefore release the drug in the intestines. Softgel delivery systems can also incorporate phospholipids or polymers or natural gums to entrap a composition, for example, a prebiotic composition, in the gelatin layer with an outer coating to give desired delayed/control release effects, such as an enteric coating.

In one embodiment, a composition is provided in a dosage form which comprises an effective amount of a bacterial spore population and one or more release controlling excipients as described herein. Suitable modified release dosage vehicles include, but are not limited to, hydrophilic or hydrophobic matrix devices, water-soluble separating layer coatings, enteric coatings, osmotic devices, multi-particulate devices, and combinations thereof. In one embodiment the dosage form is a tablet, caplet, capsule, or lollipop. In another embodiment, the dosage form is a liquid, oral suspension, oral solution, or oral syrup. In yet another embodiment, the dosage form is a gel capsule, soft gelatin capsule, or hard gelatin capsule. In another embodiment a composition comprising a bacterial spore population is provided in effervescent dosage forms. The compositions can also comprise non-release controlling excipients.

In another embodiment, a composition comprising a bacterial spore composition, optionally with a prebiotic material, is provided in the form of enteric-coated pellets, for oral administration. The compositions can further comprise glyceryl monostearate 40-50, hydroxypropyl cellulose, hypromellose, magnesium stearate, methacrylic acid copolymer type C, polysorbate 80, sugar spheres, talc, and triethyl citrate. In one embodiment a composition comprising a bacterial spore population is provided in the form of enteric-coated granules, for oral administration. The compositions can further comprise carnauba wax, crospovidone, diacetylated monoglycerides, ethylcellulose, hydroxypropyl cellulose, hypromellose phthalate, magnesium stearate, mannitol, sodium hydroxide, sodium stearyl fumarate, talc, titanium dioxide, and yellow ferric oxide.

In one embodiment, compositions can be formulated in various dosage forms for oral administration. The compositions can also be formulated as a modified release dosage form, including immediate-, delayed-, extended-, prolonged-, sustained-, pulsatile-, controlled-, extended, accelerated-, fast-, targeted-, programmed-release, and gastric retention dosage forms. These dosage forms can be prepared according to known methods and techniques (see, Remington: The Science and Practice of Pharmacy, supra; Modified-Release Drug Delivery Technology, Rathbone et al, Eds., Drugs and the Pharmaceutical Science, Marcel Dekker, Inc.: New York, N.Y., 2002; Vol. 126, which is herein incorporated by reference in its entirety). In one embodiment, the compositions are in one or more dosage forms. For example, a composition can be administered in a solid or liquid form. Examples of solid dosage forms include but are not limited to discrete units in capsules or tablets, as a powder or granule, or present in a tablet conventionally formed by compression molding. Such compressed tablets can be prepared by compressing in a suitable machine the three or more agents and a pharmaceutically acceptable carrier. The molded tablets can be optionally coated or scored, having indicia inscribed thereon and can be so formulated as to cause immediate, substantially immediate, slow, controlled or extended release of a composition comprising a prebiotic. Furthermore, dosage forms of the invention can comprise acceptable carriers or salts known in the art, such as those described in the Handbook of Pharmaceutical Excipients, American Pharmaceutical Association (1986), incorporated by reference herein in its entirety.

The compositions described herein can optionally be in liquid form. The liquid formulations can comprise, for example, an agent in water-in-solution and/or suspension form; and a vehicle comprising polyethoxylated castor oil, alcohol, and/or a polyoxyethylated sorbitan mono-oleate with or without flavoring. Each dosage form comprises an effective amount of an active agent and can optionally comprise pharmaceutically inert agents, such as conventional excipients, vehicles, fillers, binders, disintegrants, pH adjusting substances, buffer, solvents, solubilizing agents, sweeteners, coloring agents, and any other inactive agents that can be included in pharmaceutical dosage forms for oral administration. Examples of such vehicles and additives can be found in Remington's Pharmaceutical Sciences, 17th edition (1985).

The compositions are capable of being consumed ad libitum. In instances wherein a dysbiosis caused by a disease, disorder, condition or event is being addressed by administration of the compositions, the total duration of consumption, can be from about one week to about 52 weeks, or about four weeks to about twenty six weeks, or about four weeks to about twelve weeks, or about six weeks. In one embodiment, a bacterial spore composition can also be administered in combination with another substance (e.g., an antibiotic), as described herein. In one embodiment, the total duration of treatment is about 5 days to about 35 days. In one embodiment, the total duration of treatment is about 7 days to about 90 days, or about 7 days to about 60 days, or about 14 days to about 50 days, or about 14 days to about 40 days, or about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 days. In another embodiment, the total duration of treatment is about 30 days. In another embodiment, the total duration of treatment is about 34 days. In another embodiment, the total duration of treatment is about 36 days. In another embodiment, the total duration of treatment is about 38 days. In another embodiment, the total duration of treatment is about 42 days. In another embodiment, the total duration of treatment is about 60 days. In another embodiment, the total duration of treatment is about 90 days. In another embodiment, one course of therapy may be followed by another, such as an induction regimen followed by a maintenance regimen.

Animal Models

Therapeutic compositions and methods provided herein can be tested in animal models of colitis such as those known in the art. At least 66 different types of animal models have been described (Mizoguchi, 2012, Prog Mol Biol Transl Sci 105:263-320), including a dextran sodium sulfate (DSS) model and a trinitrobenzene sulfonate (TNBS) model. A candidate therapeutic composition and/or method is tested by administering the composition to the animal model either prior to induction of disease signs or symptoms, during induction, or after manifestation of at least one sign or symptom in the animal. In methods involving a pretreatment and or concurrent treatment with an antibiotic, the pretreatment may be administered prior to induction, during induction, or after the manifestation of one or more signs or symptoms. The Examples (infra) provide additional guidance for such testing.

Efficacy Measures

Efficacy of a treatment can be determined by evaluating signs and or symptoms and according to whether induction of improvement and/or maintenance of a remission or improved condition is achieved, e.g., for at least 1 week, at least two weeks, at least three weeks, at least four weeks, at least 8 weeks, or at least 12 weeks. For example, mucosal healing as judged endoscopically, histologically or via imaging techniques can be used for such evaluations, particularly for predicting long term clinical outcome in subject's diagnosed with a colitis, e.g., Crohn's disease or ulcerative colitis. Remission or signs or symptoms can be determined using clinical indices such as, for Crohn's disease, the Crohn's Disease Activity Index (CDAI), the PCDAI, or the amelioration or one or more elements of the PCDAI or CDAI, e.g., number of liquid or soft stools, abdominal pain, general well-being, presence of complications (such as arthralgia or arthritis, uveitis; inflammation of the iris; presence of erythema nodosum, pyoderma gangrenosum, or aphthous ulcers; anal fissures, fistulae, or abscesses; other fistulae, or fever), taking opiates or diphenoxylate/atropine for diarrhea, presence of an abdominal mass, hematocrit of <0.47 (males) or <0.42 (females); or percentage deviation from standard weight. In some embodiments a subject treated according to a method described herein attains and/or remains at a CDAI below 150. In some embodiments, a positive response to a method is a reduction of a subject's CDAI by at least 70 points.

For ulcerative colitis, indications of therapeutic efficacy include, for example, normalization of stool frequency, lack or urgency and absence of blood in stools. Remission is considered achieved if at least one sign or symptom is reduced for at least four weeks after completion of the treatment. Mucosal healing is one example of a measure of clinical remission. Other signs/symptoms can include normalization of C-reactive protein and/or other acute phase indicators, and subjective indicia such as those related to quality of life. Other examples of indicia can include improvement from moderate to mild using the Montreal Classification, the Mayo Score (with or without endoscopy subscore), or the Pediatric Ulcerative Colitis Index.

In general, methods and compositions described herein are useful for treating a subject diagnosed with a colitis.

Other indicators of efficacy of a therapeutic composition and/or method for treating a colitis include engraftment of at least one bacterial OTU identified in the BSC component of the therapeutic composition, at 7 days, for example, engraftment or at least one keystone bacterial OTU; engraftment of at least one bacterial OTU at 2 weeks, 3 weeks, 4 weeks, 5 weeks, 6 weeks, 7 weeks, 8 weeks, 9 weeks, 10 weeks, 11 weeks, or 12 weeks; clinical remission at 4 weeks, 5 weeks, 6 weeks, 7 weeks, 8 weeks, 9 weeks, 10 weeks, 11 weeks, or 12 weeks (e.g., a Mayo score<=2 with no subscore>1); or endoscopic remission at 4 weeks (Mayo endoscopy score of 0). Keystone OTUs have been described in PCT/US2014/030817 (WO 2014/145958).

### Definitions

A “therapeutically effective amount” of a therapeutic composition described herein can vary according to factors such as the disease state, age, sex, and weight of the individual, and the ability of the compound to elicit a desired response in the individual, e.g., amelioration of at least one disorder parameter, or amelioration of at least one symptom of the disorder (and optionally, the effect of any additional agents being administered). A therapeutically effective amount is also one in which any toxic or detrimental effects of the composition are outweighed by the therapeutically beneficial effects. A composition as described herein is generally administered in a therapeutically effective amount.

It is also understood that there may be a range of the therapeutically effective amount of the individual components of the therapeutic composition (BSC and antibiotic).

### Equivalents

All technical features can be individually combined in all possible combinations of such features.

The invention may be embodied in other specific forms without departing from the spirit or essential characteristics thereof. The foregoing embodiments are therefore to be considered in all respects illustrative rather than limiting on the invention described herein.

### EXAMPLES

The following non-limiting examples further illustrate embodiments of the inventions described herein.

### Example 1

### Method of Making a BSC

A BSC was prepared using stool specimens obtained from healthy human donors. Stool samples were fractionated, resulting in a preparation of Firmicutes spores. Briefly, fresh stool specimens were collected and then frozen at −80° C. Approximately 150 g was suspended and homogenized in normal saline and filtered through mesh screens. The resulting slurry was centrifuged, the supernatant containing bacterial cells and spores was collected and, using 100% ethanol, brought to 50% (wt/wt). The ethanol preparation was incubated at room temperature for one hour, pelleted by centrifugation, washed with saline to remove ethanol, and resuspended in sterile glycerol producing a BSC. The BSC was stored at −80° C. until ready for use.

The BSC was characterized for spore concentration and absence of residual gram-negative bacteria. Spore content was determined by measuring the dipicolinic acid (DPA) content and normalizing against the DPA content of known numbers of spores representing three commensal species (Hindle and Hall. 1999 Analyst 124:1599-604). The absence of residual gram-negative bacteria was confirmed by selective plating on MacConkey lactose agar and Bacteroides bile esculin agar. No vegetative microbes were found in any BSC preparation within the limit of assay detection (<30 colony-forming units/mL).

### Example 2

### Dextran Sodium Sulfate (DSS) Model of Colitis With or Without a Broad-Spectrum Antibiotic Pretreatment

The DSS model is a well-characterized model of colitis, used as a model for inflammatory bowel disease (IBD), including, e.g., ulcerative colitis (Wirtz, 2007 Nat Protoc 2:541-6). In this model, DSS is delivered in the drinking water and induces colitis through direct toxicity to basal crypt cells and causes mucosal disruption with ensuing innate immune activation. DSS-induced inflammation is restricted to the large intestine, and the pathology is independent of adaptive immunity. A BSC was tested in this model to determine the ability of a healthy microbiome to ameliorate these features of IBD, e.g., ulcerative colitis expressed in the DSS model.

Briefly, three-week-old male C57B1/6 mice, 15 per group (10 in the naïve control group), were treated according to Table 1.

TABLE 1

| | | | Antibiotic | DSS Disease | Test Item and |
|---|---|---|---|---|---|
| Group | Group | Group | Pretreatment | Induction | Day of |
| Number | Size (n) | Description | Days 0-9¹ | Days 31-35² | Initiation³ |
| 1 | 10 | Naïve Control | None | None | None |
| 2 | 15 | Negative | None | + | PBS⁴ |
| | | Control | | | Day 10 |
| 3 | 15 | Positive Control | None | + | Budesonide |
| | | | | | Day 24 |
| 4 | 15 | BSC⁵ | None | + | BSC |
| | | | | | Day 0 |
| 5 | 15 | Abx⁶ Control | + | + | PBS |
| | | | | | Day 10 |
| 6 | 15 | Abx + SPF⁷ | + | + | SPF cecal slurry |
| | | | | | Day 10 |
| 7 | 15 | Abx + BSC | + | + | BSC |
| | | | | | Day 10 |

¹Ad libitum oral administration of antibiotic mixture in drinking water
²Ad libitum oral administration of 2% DSS in drinking water
³Therapies were administered by oral gavage 3 times a week until Day 41 starting on the day indicated
⁴PBS = phosphate-buffered saline
⁵BSC = Research grade BSC was produced in a pilot scale manufacturing process in a laboratory environment without special precaution to ensure aseptic, closed operations. Research grade material is representative of clinical grade material and contains the active spore component of clinical process BSC. About 1e7 spores per dose.
⁶Abx = antibiotic cocktail (0.5 mg/ml kanamycin, 0.044 mg/ml gentamycin, 1062.5 U/ml colistin, 0.269 mg/ml metronidazole, 0.156 mg/ml ciprofloxacin, 0.1 mg/ml ampicillin, and 0.056 mg/ml vancomycin.
⁷SPF = cecal content slurry from specific-pathogen-free mice in PBS. About 1e8 organisms per dose.

An objective of this experiment was to evaluate the effect of pretreatment with an antibiotic cocktail on the efficacy of a BSC in treating IBD, e.g., UC. Accordingly, three groups received a 10-day course of an antibiotic cocktail in drinking water prior to receiving their treatment (Groups 5-7). Mice in Groups 2 and 4-7 were dosed by oral gavage three times per week with either phosphate-buffered saline (PBS) (control Groups 2 and 5), a BSC (Groups 4 and 7), or mouse cecal slurry (Group 6). On Days 31-35, mice were exposed to 2% DSS in their drinking water (Groups 2-7), and were then observed until Day 41. Periodic measurements included body weight and clinical scores. On Day 41, animals were euthanized and features of the colon were evaluated including weight, length, and gross pathology. Colon samples were prepared for histopathology and scored by a pathologist blinded to treatment group and the nature of the test group.

Overall, DSS treatment resulted in significant body weight loss, worsened clinical and gross pathology scores, and reduced colon length compared to naïve mice (Group 2 vs. Group 1). No statistically significant changes in any of the disease parameters were observed in mice receiving BSC alone (Group 4) or budesonide (Group 3; the glucocorticoid Positive Control), except for a small, but significant improvement in clinical score with budesonide treatment (Group 3) compared to the Negative Control mice receiving PBS in the absence of antibiotic pretreatment (Group 2).

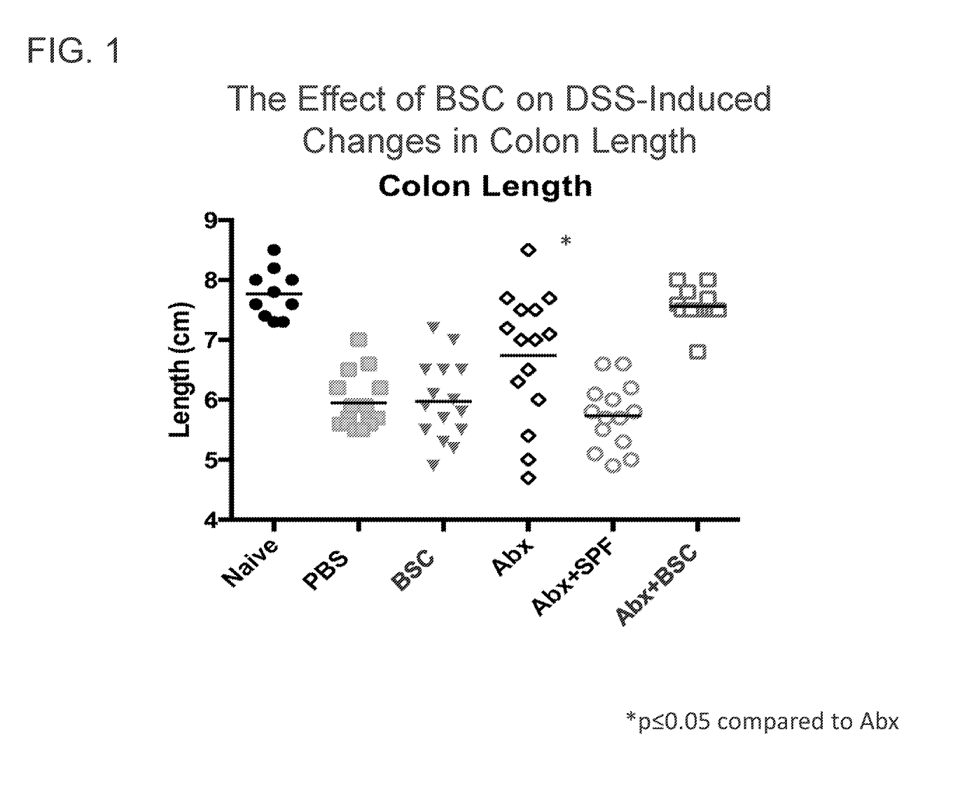

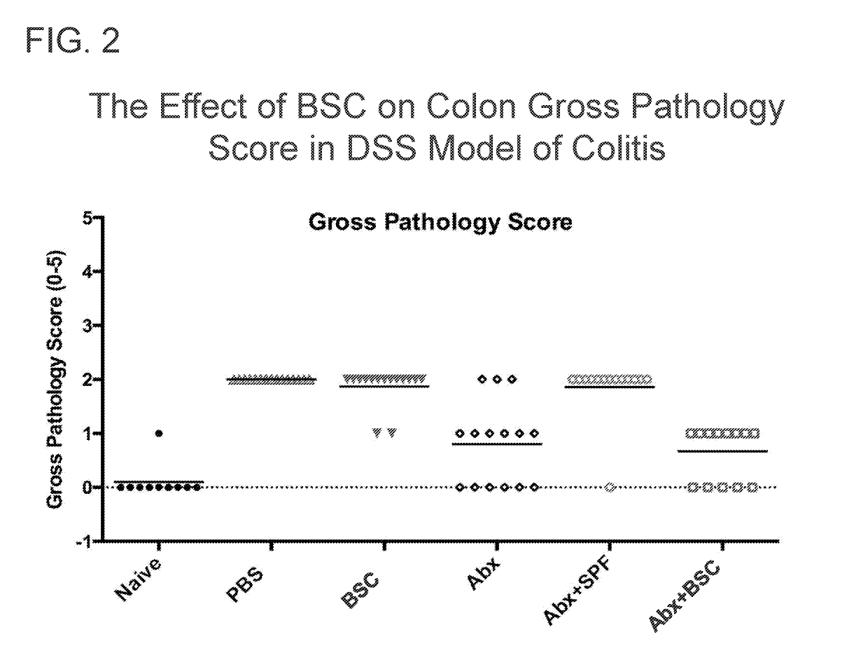

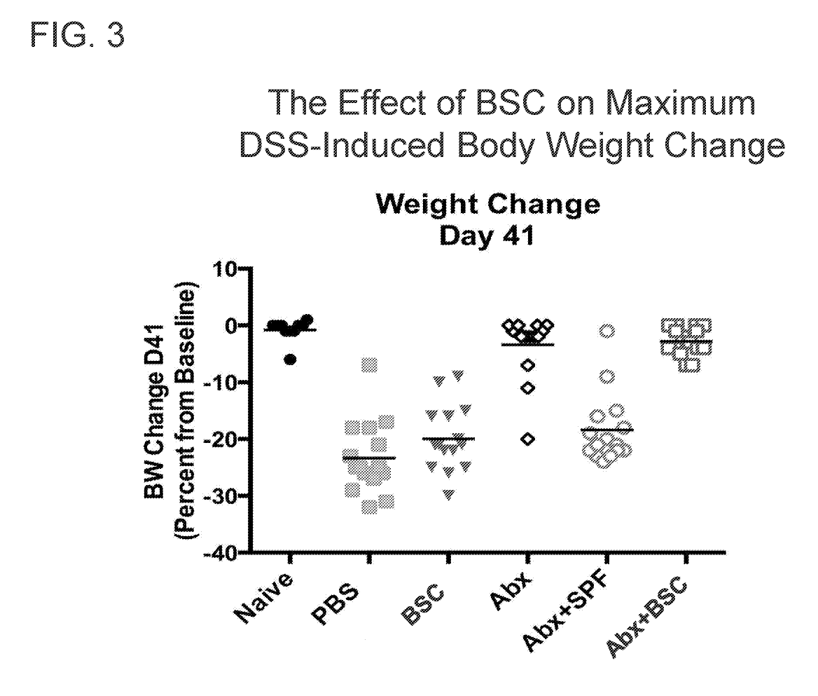

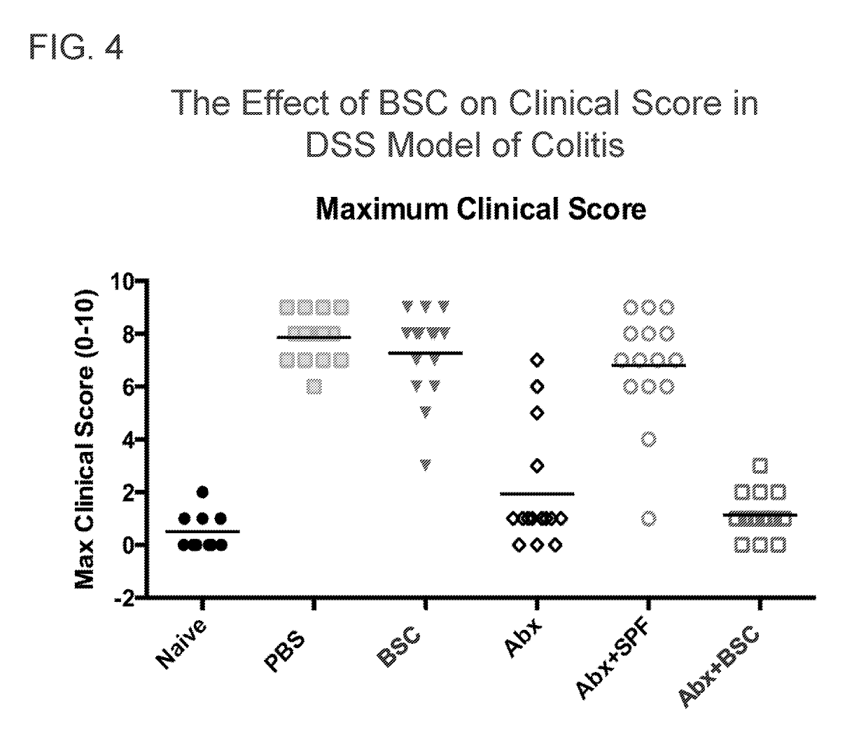

Mice pretreated with a broad-spectrum antibiotic cocktail and administered PBS (Abx Control, Group 5) were largely protected against DSS pathology compared to the Negative Control group. Mice orally inoculated with SPF mouse cecal material after the antibiotic cocktail (Abx+SPF, Group 6) lost this protection and exhibited DSS sensitivity comparable to the Negative Control group. Abx+BSC (Group 7)-treated mice were protected against DSS-induced disease similarly to antibiotic treatment alone (Abx Control, Group 5). However, there were indications of further protection against colon pathology based on increased colon length in the Abx+BSC (Group 7) compared to antibiotic treatment alone (Group 5) (FIG. 1). In general, shortening of the colon is indicative of inflammation. While there were no significant differences overall in colon histopathology scores between the Abx+PBS and the Abx+BSC treatments, the treatment effect was more consistent in the Abx+BSC cohort (FIG. 2). A similar effect of consistency within the cohort was observed for body weight change (Abx+BSC had a more consistent protective effect than Abx+PBS) (FIG. 3). There were non-significant trends toward reduced percent body weight loss, lower clinical score, and lower gross pathology score in the Abx+BSC group following DSS treatment (FIG. 4).

In summary, these data demonstrate that DSS treatment resulted in body weight loss, increased clinical score, colon shortening, and inflammation. In the absence of antibiotic pretreatment, budesonide and BSC treatment alone provided little or no protection against pathology. Pretreatment with a broad-spectrum antibiotic prevented the DSS-induced disease; however, inoculation with cecal contents from normal mice restored the sensitivity to DSS pathology. Both PBS and BSC treatment administered after antibiotic pretreatment (Groups 5 and 7) were efficacious in preventing DSS-induced disease. In addition, BSC provided improvement above the protection afforded by the antibiotics alone as evidenced by reduced animal-to-animal variability and longer colon length within the group. These data indicate that BSC in combination with an antibiotic pretreatment is protective in the mouse DSS experimental colitis model.

### Example 3

### DSS Model of Colitis With or Without Vancomycin Pretreatment

To further examine the effect of antibiotic and a BSC on ulcerative colitis, the DSS model described supra was used with the addition of arms evaluating vancomycin as the sole antibiotic. In this experiment, three-week-old male C57B1/6 mice, 15 per group (9 in the Naïve control group), were treated according to the Table 2.

TABLE 2

| | | | | DSS | |
|---|---|---|---|---|---|
| | | | Antibiotic | Disease | Test Item and |
| Group | Group | Group | Pretreatment | Induction | Day of |
| Number | Size (n) | Description | Days 0-9¹ | Days 28-32² | Initiation³ |
| 1 | 9 | Naïve Control | None | None | None |
| 2 | 15 | Negative | None | + | PBS⁴ |
| | | Control | | | Day 9 |
| 3 | 15 | Positive | None | + | Anti-IL12⁵ |
| | | Control | | | Day 28 |
| 4 | 15 | BSC⁶ | None | + | BSC |
| | | | | | Day 0 |
| 5 | 15 | Vanco⁷ | Vancomycin | + | PBS |
| | | Control | | | Day 9 |
| 6 | 15 | Vanco + BSC | Vancomycin | + | BSC |
| | | | | | Day 9 |
| 7 | 15 | Abx⁸ Control | Antibiotic Mixture | + | PBS |
| | | | | | Day 9 |
| 8 | 15 | Abx + BSC | Antibiotic Mixture | + | BSC |
| | | | | | Day 9 |

¹Ad libitum oral administration of antibiotic mixture or vancomycin alone in drinking water
²Ad libitum oral administration of 2% DSS in drinking water
³Therapies were administered by oral gavage 3 times a week until Day 38 starting on the day indicated
⁴PBS = phosphate-buffered saline
⁵Anti-IL12 = anti-interleukin 12 p40 subunit antibody and was administered intraperitoneally one time on Day 28
⁶BSC = Research grade BSC was produced in a pilot scale manufacturing process in a laboratory environment without special precaution to ensure aseptic, closed operations. Research grade material is representative of clinical grade material and contains the active spore component of a clinical BSC.
⁷Vanco = vancomycin
⁸Abx = antibiotic cocktail (0.5 mg/ml kanamycin, 0.044 mg/ml gentamycin, 1062.5 U/ml colistin, 0.269 mg/ml metronidazole, 0.156 mg/ml ciprofloxacin, 0.1 mg/ml ampicillin, and 0.056 mg/ml vancomycin

Either vancomycin (Groups 5 and 6) or an antibiotic mixture (Groups 7 and 8) was administered to mice in their drinking water prior to receiving treatment to evaluate the effect on BSC efficacy. Mice were dosed by oral gavage with either PBS (Groups 2, 5, and 7) or BSC (Groups 6 and 8) three times per week starting on Day 9 and continuing through the end of the study (Day 38). Group 4 was dosed with a BSC three times per week starting on Day 0. On days 28-32, mice were exposed to 2% DSS in their drinking water (Groups 2-8) and observed until Day 38. Periodic measurements included body weight and clinical scores. On Day 38, animals were euthanized and features of the colon were evaluated including weight, length, and gross pathology. Colon samples were prepared for histopathology and scored by a pathologist blinded to treatment group and the nature of the Test Item.

Overall, DSS treatment resulted in significant body weight loss, worsened clinical and gross pathology scores, thickening of the colon, decreased colon length, and increased colon inflammation and edema (Negative Control Group 2 vs. Naïve Group 1). The Positive Control, anti-IL-12 p40 (Group 3), showed signals of activity with improvements in body weight and clinical score beginning on Days 36-38 and overall reduced maximum clinical and histopathology scores as compared to the Negative Controls.

Compared to the Negative Controls, animals receiving BSC alone (Group 4) exhibited more severe maximal body weight loss (15.5% vs. 22.1%) following DSS induction. However, there were no differences in clinical scores, colon lengths, gross pathology, and histopathology score at study termination on Day 38. Body weight changes and clinical symptoms were not observed during the 3 week BSC treatment prior to addition of DSS to drinking water.

Pretreatment with vancomycin (Group 5) provided significant protection against DSS-induced disease. The maximum weight loss in the vancomycin group (Group 5) was significantly reduced compared to the Negative Control group. Vancomycin (Group 5) resulted in reduced clinical scores and overall improvement in colon pathology as evidenced by increased colon length, reduced colon weight, and improved gross pathology and histopathology scores compared to the Negative Control (Group 2).

Vanco+BSC (Group 6) protected against body weight loss to the same extent as Vanco Control (Group 6). However, the, Vanco+BSC treatment provided benefit in multiple measures compared to vancomycin alone. Mice receiving Vanco+BSC exhibited improvement of clinical scores and lower colon weights compared to the Vanco alone cohort (Group 5). Gross pathology scores in the Vanco+BSC group were higher than for the Vanco only group, but there was no difference in blinded microscopic histopathology scores.

Similar to the Vanco Control group, the Abx Control (Group 7) cohort exhibited reduced body weight loss, improved clinical scores, and reduced colon pathology in response to DSS treatment compared to the Negative Control (Group 2). Abx in combination with BSC (Group 8) resulted in similar outcomes relative to Abx alone (Group 7) with the exception that treatment with Abx+BSC (Group 8) resulted in reduced colon weight. The observation of reduced colon weight is consistent with a similar effect observed comparing Vanco+BSC to the Vanco only control.

In summary, these data demonstrate that DSS treatment resulted in body weight loss, increased clinical score, colon shortening, and inflammation. The Positive Control, anti-IL-12 p40 (Group 3), showed signals of activity with improvements in body weight and clinical score beginning on Days 36-38 and overall reduced maximum clinical and histopathology scores as compared to the Negative Controls. In the absence of antibiotic pretreatment, BSC treatment alone was well-tolerated as evidenced by the absence of body weight changes and clinical symptoms during the 3 week BSC treatment prior to addition of DSS to drinking water. However, BSC alone treatment had no protective effect against DSS-induced pathology. Pretreatment with vancomycin or a broad-spectrum antibiotic mixture largely protected against DSS-induced disease, but these cohorts exhibited worsening in colon pathology as evidenced by changes in gross pathology and histopathology scores compared to naïve mice. The administration of BSC following vancomycin pretreatment was also protective against DSS-induced disease and was significantly more efficacious than Vanco based on improvement in clinical score and colon weight, and non-significant trends towards reduced maximum body weight loss, increased colon length and reduced histopathology. While treatment with BSC after the broad-spectrum antibiotic mixture did not provide an elevated level of protection in the DSS-induced over its control group in most measures, colon weight was reduced when BSC was combined with antibiotic treatment. Taken together, these data indicate that treatment with BSC following vancomycin is protective in the mouse DSS experimental colitis model and is therefore useful as a treatment regime for treating an IBD, e.g., ulcerative colitis.

### Example 4

### TNBS Model of Colitis With and Without a Broad-Spectrum Antibiotic Pretreatment

Trinitrobenzene sulfonic acid (TNBS), also called picrylsulfonic acid, is a frequently used chemical inducer of inflammatory bowel disease (Wirtz, 2007, supra). To obtain TNBS-induced colitis, TNBS is prepared in ethanol and administered directly into the colon. The combination of ethanol and TNBS results in disruption of the epithelial mucosal layer and haptenization of autologous and microbial proteins, which increases their immunogenicity and induces an adaptive, Th1-type immune response. This model allows evaluation of therapeutics involved in protection against epithelial damage in addition to modulators of adaptive immunity.

To test the efficacy of BSC in this model, three-week-old female Balb/c mice, 18 per group (10 in the naïve control group), were treated according to Table 3.

TABLE 3

| | | | | TNBS | Treatment |
|---|---|---|---|---|---|
| | | | Antibiotic | Disease | and |
| Group | Group | Group | Pretreatment | Induction | Day of |
| Number | Size (n) | Description | Days 0-9¹ | Days 31² | Initiation³ |
| 1 | 10 | Naïve | None | None | None |
| | | Control | | | |
| 2 | 18 | Negative | None | + | PBS⁴ |
| | | Control | | | Day 10 |
| 3 | 18 | Positive | None | + | Budesonide |
| | | Control | | | Day 24 |
| 4 | 18 | BSC⁵ | None | + | BSC |
| | | | | | Day 0 |
| 5 | 18 | Abx⁶ Control | + | + | PBS |
| | | | | | Day 10 |
| 6 | 18 | Abx + SPF⁷ | + | + | SPF cecal |
| | | | | | slurry |
| | | | | | Day 10 |
| 7 | 18 | Abx + BSC | + | + | BSC |
| | | | | | Day 10 |

¹Ad libitum oral administration of antibiotic mixture in drinking water
²Intracolonic administration of 80 μl of 2% TNBS
³Therapies were administered by oral gavage 3 times a week until Day 38 starting on the day indicated
⁴PBS = phosphate-buffered saline
⁵BSC = Research grade BSC was produced in a pilot scale manufacturing process in a laboratory environment without special precaution to ensure aseptic, closed operations. Research grade material is representative of clinical grade material and contains the active spore component of clinical BSC.
⁶Abx = antibiotics
⁷SPF = microbiota prepared from the cecal contents of specific pathogen free (SPF) mice

An objective of the experiment was to evaluate the effect of antibiotic pretreatment on BSC efficacy, therefore three groups received a 10-day course of an antibiotic cocktail in drinking water prior to receiving their treatment (Groups 5-7). Mice in Groups 2 and 4-7 were dosed by oral gavage three times per week with either phosphate-buffered saline (PBS) (control Groups 2 and 5), BSC (Groups 4 and 7), or mouse cecal slurry (Group 6). On Day 31, mice were administered intracolonic 2% TNBS (Groups 2-7), and were then observed until Day 38. Periodic measurements included body weight and clinical scores. On Day 38, animals were euthanized and features of the colon were evaluated to generate an overall gross pathology score. Colon samples were prepared for histopathology and scored by a pathologist blinded to treatment group and the nature of the Test Item.

Death following TNBS induction was the most striking outcome. The loss of greater than 50% of the animals suggests that an over dosage of TNBS occurred. In addition, the use of xylazine and ketamine for anesthesia at the time of TNBS instillation may have enhanced the severity of disease. This finding is consistent with reports in the literature (Scheiffele and Fuss, 2002 Curr Protocols Immunol. Unit 15.19. Published Online: 1 Aug. 2002, DOI: 10.1002/0471142735.im1519s49). The kinetics of mortality did not appear to differ among the treatment groups (Groups 2-7). Mortality ranged from 39% (7/18 dead in Negative Control Group 2 and BSC Group 4) to 61% (11/18 dead in Positive Control Group 3). The Abx Control Group 5 had an intermediate level of mortality at 56% (10/18 dead). There were no deaths in the Naïve Group. Death rates for the groups receiving BSC (Groups 4 and 7) did not differ from any other treatment arm.

TNBS treatment also resulted in significant body weight loss, worsened clinical and gross pathology scores, and death compared to naïve mice (Group 2 vs. Group 1). Budesonide (Group 3), the glucocorticoid Positive Control, did not significantly protect mice from TNBS-induced disease with respect to any of the parameters examined. Rather, the Positive Control group lost significantly more weight than the Negative Control mice and had worsened clinical scores. Mice that were administered BSC alone (Group 4) did not differ from the Negative Control mice in body weight, clinical scores, or gross pathology and histology scores. The Abx Controls (Group 5) exhibited significantly greater maximal body weight loss and worsened clinical scores compared to the Negative Controls (Group 2). Overall, the Abx+SPF (Group 6) cohort performed similarly to their Abx Controls (Group 5) with the exception of improved clinical scores.

In contrast to the other treatment groups with minimal efficacy, the Abx+BSC (Group 7) had consistent signs of improvement. Abx+BSC (Group 7) lost significantly less weight on Day 36 and overall, had lower maximal body weight loss than their controls (Abx Control Group 5). While not statistically significant, the Abx+BSC (Group 7) had clinical scores that were among the lowest of the treatment groups and were significantly lower than their controls (Abx Control Group 5). Abx+BSC (Group 7) also showed a strong trend toward protection with respect to gross pathology (p=0.1). The Abx+BSC mice had the lowest gross pathology score of any treatment group. No significant differences were observed for any of the treatment groups with respect to histopathology scores. Thus, it appears that the addition of BSC to the antibiotic mixture pretreatment had a beneficial effect upon body weight, clinical scores and colon gross pathology.

In summary, these data are consistent with an efficacy signal of BSC delivered after antibiotic treatment in the TNBS colitis model despite the observation that these data are in a setting in which there was a high level of mortality and corresponding significant clinical symptoms and tissue pathology, indicating an overdosing of animals with TNBS, possibly as a result of anesthesia. In some embodiments a subject diagnosed with IBD or at risk for a flare of IBD is treated with a combination of antibiotic(s) and a BSC.

### Example 5

### TNBS Model of Colitis With and Without a Broad-Spectrum Antibiotic or Vancomycin Pretreatment

Additional experiments were carried out using the TNBS model (described supra) using a BSC in combination with an antibiotic. In these experiments, three-week-old male Balb/c mice, 24 per group (9 in the naïve control group), were treated according to Table 4.

TABLE 4

| | | | | TNBS | Test |
|---|---|---|---|---|---|
| | | | Antibiotic | Disease | Item and |
| Group | Group | Group | Pretreatment | Induction | Day of |
| Number | Size (n) | Description | Days-1-5¹ | Days 28² | Initiation³ |
| 1 | 9 | Naïve Control | None | None | None |
| 2 | 24 | Negative | None | + | PBS⁴ |
| | | Control | | | Day 7 |
| 3 | 24 | Positive | None | + | Anti- |
| | | Control | | | IL12⁵ |
| | | | | | Day 28 |
| 4 | 24 | BSC⁶ | None | + | BSC |
| | | | | | Day 0 |
| 5 | 24 | Vanco⁷ Control | Vancomycin | + | PBS |
| | | | | | Day 7 |
| 6 | 24 | Vanco + BSC | Vancomycin | + | BSC |
| | | | | | Day 7 |
| 7 | 24 | Abx⁸ Control | Antibiotic | + | PBS |
| | | | Mixture | | Day 7 |
| 8 | 24 | Abx + BSC | Antibiotic | + | BSC |
| | | | Mixture | | Day 7 |

¹Ad libitum oral administration of antibiotic mixture or vancomycin alone in drinking water
²Intracolonic administration of 80 μl of 2% TNBS
³Therapies were administered by oral gavage 3 times a week until Day 35 starting on the day indicated, except where indicated.
⁴PBS = phosphate-buffered saline
⁵Anti-IL12 = antibody against interleukin 12 p40 subunit, 25 mg/kg was administered intraperitoneally one time on Day 28
⁶BSC = a research grade BSC was produced in a pilot scale manufacturing process in a laboratory environment without special precaution to ensure aseptic, closed operations. Research grade material is representative of clinical grade material and contains the active spore component of clinical BSC.
⁷Vanco = vancomycin
⁸Abx = antibiotics

Either vancomycin (Groups 5 and 6) or an antibiotic mixture (Groups 7 and 8) was administered to mice in their drinking water prior to receiving treatment to evaluate the effect on BSC efficacy (Groups 5-8). Mice were dosed by oral gavage with either PBS (Groups 2, 5, and 7) or BSC (Groups 6 and 8) three times per week starting on Day 7 and continuing through the end of the study (Day 35). Mice in group 4 received BSC three times per week by oral gavage starting on Day 0. On day 28, mice were anesthetized with isoflurane and received intracolonic 2% TNBS (Groups 2-8). Periodic measurements included body weight and clinical scores. On Day 35, animals were euthanized and gross pathology was evaluated. Colon samples were prepared for histopathology and scored by a pathologist blinded to treatment group and the nature of the Test Item.

Overall, TNBS treatment resulted in significant body weight loss, mortality, and worsened clinical and gross pathology (Negative Control Group 2 vs. Naïve Group 1). Mortality following TNBS induction was highest in the Negative Control group at 33% (8/24 dead or euthanized). There were no deaths in the Naïve Control Group 1, Abx Control Group 7 or Abx+BSC Group 8. Intermediate levels of mortality were seen in the Positive Control Group 3 (1/24, 4%), BSC Group 4 (1/24, 4%), Vanco Control Group 5 (2/24, 8%), and Vanco+BSC Group 6 (3/24, 12%). A comparison of all the survival curves found significant differences in the distributions of deaths among treatment groups (log-rank (Mantel-Cox) test, p=0.02). Further pairwise analysis showed a strong trend in decreased survival in the Negative Control Group 2 compared to Naïve Group 1 (p=0.06) with no other groups differing from one another. Thus, the observed differences in survival curves were attributable to the higher rate of death in the Negative Control group.

Compared to the Naïve Group 1 cohort, mice in the Negative Control/Group 2 exhibited significant weight loss. This body weight loss followed TNBS administration on Day 28 and was maintained through Day 35 (termination). During Days 31-33, mice in the Positive Control Group 3, BSC Group 4, and the Abx Control Group 7 all gained significantly more weight than the Negative Control mice. Analyzing maximal percent weight loss, the BSC alone (Group 4) also showed a trend toward improvement compared to the Negative Control mice (p=0.10).

While mice pretreated with the antibiotic mixture alone (Abx Control Group 7) exhibited protection against weight loss, there was no effect of vancomycin alone (Vanco Control Group 5). There was no significant increase in body weight when BSC was administered following vancomycin or antibiotic pretreatment (Groups 6 and 8) as compared to their respective control groups (Groups 5 and 7). Overall, there were no significant effects on maximum percent body weight loss for any of the groups receiving vancomycin or the antibiotic mixture when compared to the Negative Control/Group 2.

Clinical scores for the Negative Control group were significantly worsened by TNBS treatment compared to the Naïve group. Clinical scores were improved for the Positive Control Group 3, BSC Group 4, and Abx Control Group 7 as all were significantly lower than the Negative Control group. Vanco Control Group 5 was not statistically improved relative to the Negative Control Group 2; however, BSC in combination with vancomycin (Group 6) had improved clinical score relative to the Vanco Control Group 5, showing an added benefit with the combination treatment. No significant differences in clinical scores were observed between the Abx Control group and Abx+BSC. Thus, both the anti-IL-12 positive control and BSC alone improved the clinical signs and symptoms of TNBS colitis. BSC following vancomycin also showed improvement compared to the vancomycin-only control.

As expected, TNBS treatment resulted in significantly increased gross pathology scores in the Negative Control Group 2 compared to the Naïve Group 1. Congruent with decreased body weight loss and improved clinical scores, the colon gross pathology score was significantly improved in the BSC alone (Group 4). Histopathology scores were also consistent with the other endpoints with the Positive Control Group 2 having significant histological signs of disease and the BSC Group 4 having a strong trend of improvement (p=0.14). While the Vanco Group was not different than the Negative Control Group 2, there was a significant improvement in histopathology with the combination Vanco+BSC group (Group 6) compared to Vanco alone (Group 5). No additional significant differences were noted among the groups.

In summary, TNBS-induced disease was characterized by weight loss, mortality, and worsened clinical and gross pathology scores. The group receiving BSC alone was significantly improved in three parameters measured (body weights, clinical score, and gross pathology score) with a strong trend toward improvement in three others (mortality, maximum body weight loss, and histopathology). In addition, the combination of BSC with vancomycin significantly improved clinical and histopathology scores as compared to its control group of vancomycin only. In contrast, the significant level of protection that was afforded by the antibiotic mixture alone (Abx Control Group 5) made it difficult to detect added benefit due to the addition of BSC treatment (Abx+BSC).

These data indicate that both BSC and vancomycin followed by BSC are protective in the mouse TNBS experimental colitis model.

Applicants note that in a repeat of this experiment, TNBS-induced disease was characterized by weight loss, mortality, and worsened clinical scores and colon pathology. Although rates of mortality were lower than those in the first TNBS study, they were still as high as 25% indicating that TNBS toxicity dominated the effects seen in the experiment. BSC alone or in combination with vancomycin or antibiotics did not significantly improve outcomes. Anti-IL-12 p40, had no beneficial effect upon weight, mortality, or clinical scores in this study, and was associated with an improvement in tissue damage as assessed by improved gross pathology. Because of the unexpectedly poor response in the anti-IL-12p40 control, Applicants believe the overall results of this experiment were an anomaly.

### Example 6

### Treatment of Human Subjects with Active Mild to Moderate Ulcerative Colitis

To determine the safety and possible efficacy of a BSC for treating ulcerative colitis, subjects having mild to moderate ulcerative colitis are identified (e.g., subjects determined by sigmoidoscopy to have at least 15 cm of disease, a Mayo score of ≥4 to ≤10, and a Mayo endoscopic subscore of ≥2). Subjects are pretreated with vehicle or vancomycin for 6 days then are treated as indicated in Table 5.

TABLE 5
Experimental design for treatment of human mild to moderate
ulcerative colitis

| Pretreatment | | | |
|---|---|---|---|
| (6 days) | BSC | Frequency | Duration |
| Placebo | BSC + placebo | Weekly BSC; Placebo 6 days/ | 8 weeks |
| | | week | |
| Vancomycin | BSC + placebo | Weekly BSC; Placebo 6 days/ | 8 weeks |
| | | week | |
| Vancomycin | BSC only | Daily | 8 weeks |
| Placebo | Placebo | Daily | 8 weeks |

In addition to evaluating the clinical symptoms of ulcerative colitis, changes in the composition of subjects' gastrointestinal microbiome are assessed.

Treatment with a BSC improves ulcerative colitis as shown by, e.g., a decrease from a baseline of the total Mayo score≥1 point (e.g., ≥3 points); a decrease in rectal bleeding subscore of ≥1 point or absolute rectal bleeding subscore of 0 or 1 at week 8; complete endoscopic remission as indicated by an endoscopic Mayo score of 0 or 1 at week 8; or complete remission as indicated by a total Mayo score≥2 and endoscopic subscore of 0 or 1 at week 8.

The invention is further described in the following numbered paragraphs.

1. A method of treating a subject diagnosed with a colitis, the method comprising: (a) administering an antibiotic to the subject; and (b) administering a bacterial spore composition (BSC) to the subject.

2. The method of paragraph 1, wherein the colitis is Crohn's disease or ulcerative colitis.

3. The method of paragraph 1 or 2, wherein the antibiotic is vancomycin.

4. The method of any one of paragraphs 1 to 3, wherein the antibiotic and the bacterial spore composition are administered concurrently.

5. The method of any one of paragraphs 1 to 3, wherein the antibiotic and the bacterial spore composition are administered sequentially.

6. The method of any one of paragraphs 1 to 5, wherein the bacterial spore composition is administered within 24 hours, 2 days, 3 days, 4 days, 5 days, 6 days, 7 days, 10 days, two weeks, or three weeks of the final dosing of the antibiotic.

7. The method of any one of paragraphs 1 to 6, wherein the bacterial spore composition is administered in a single dose.

8. The method of any one of paragraphs 1 to 6, wherein the bacterial spore composition is administered every day, at least every other day, at least every 3 days, at least every 4 days, at least every 5 days, at least every 6 days, at least every week, at least every 2 weeks, at least every 3 weeks, at least every 4 weeks, at least every 8 weeks, at least every 12 weeks, or at least every 16 weeks.

9. The method of any one of paragraphs 1 to 8, wherein the bacterial spore composition is in a capsule or pill.

10. The method of any one of paragraphs 1 to 9, wherein the composition comprises less than or equal to 99% vegetative cells.

11. The method of any one of paragraphs 1 to 10, wherein the subject has active colitis.

12. The method of any one of paragraphs 1 to 11, wherein the subject has been diagnosed with mild to moderate ulcerative colitis.

13. The method of any one of paragraphs 1 to 12, wherein the subject is treated with the BSC weekly for at least 8 weeks or daily for at least 8 weeks.

14. The method of any one of paragraphs 1 to 13, wherein the BSC comprises spore forming bacteria.

15. The method of any one of paragraphs 1 to 14, wherein the BSC comprises spores.

16. The method of paragraph 15, wherein the spores are directly derived from human feces.

17. The method of paragraph 16, wherein the spores are directly derived using ethanol.

18. The method of any one of paragraphs 14 to 17, wherein the composition consists essentially of spores.

19. The method of paragraph 10, wherein the composition comprises less than or equal to 20% vegetative cells.

20. Use of a bacterial spore composition, in combination with an antibiotic, for treating colitis.

21. Use of a bacterial spore composition, in combination with an antibiotic, for preparing medicaments for treating colitis.

Other embodiments are within the scope of the following claims.

What is claimed is:

1. A method of treating a subject diagnosed with a colitis, the method comprising:
(a) administering an antibiotic to the subject; and
(b) administering a bacterial spore composition (BSC) to the subject.

2. The method of claim 1, wherein the colitis is Crohn's disease or ulcerative colitis.

3. The method of claim 1, wherein the antibiotic is vancomycin.

4. The method of claim 1, wherein the antibiotic and the bacterial spore composition are administered concurrently.

5. The method of claim 1, wherein the antibiotic and the bacterial spore composition are administered sequentially.

6. The method of claim 1, wherein the bacterial spore composition is administered within 24 hours, 2 days, 3 days, 4 days, 5 days, 6 days, 7 days, 10 days, two weeks, or three weeks of the final dosing of the antibiotic.

7. The method of claim 1, wherein the bacterial spore composition is administered in a single dose.

8. The method of claim 1, wherein the bacterial spore composition is administered every day, at least every other day, at least every 3 days, at least every 4 days, at least every 5 days, at least every 6 days, at least every week, at least every 2 weeks, at least every 3 weeks, at least every 4 weeks, at least every 8 weeks, at least every 12 weeks, or at least every 16 weeks.

9. The method of claim 1, wherein the bacterial spore composition is in a capsule or pill.

10. The method of claim 1, wherein the composition comprises less than or equal to 99% vegetative cells.

11. The method of claim 1, wherein the subject has active colitis.

12. The method of claim 1, wherein the subject has been diagnosed with mild to moderate ulcerative colitis.

13. The method of claim 1, wherein the subject is treated with the BSC weekly for at least 8 weeks or daily for at least 8 weeks.

14. The method of claim 1, wherein the BSC comprises spore forming bacteria.

15. The method of claim 1, wherein the BSC comprises spores.

16. The method of claim 15, wherein the spores are directly derived from human feces.

17. The method of claim 16, wherein the spores are directly derived using ethanol.

18. The method of claim 14, wherein the composition consists essentially of spores.

19. The method of claim 10, wherein the composition comprises less than or equal to 20% vegetative cells.

20. Use of a bacterial spore composition, in combination with an antibiotic, for treating colitis.

21. Use of a bacterial spore composition, in combination with an antibiotic, for preparing medicaments for treating colitis.
